(19) United States
(12) Patent Application Publication
(10) Pub. No.: US 2021/0268046 A1
(43) Pub. Date: Sep. 2, 2021

(54) CANNABINOID & PROBIOTIC SUPPOSITORIES & METHODS OF USE

(71) Applicant: HoneyHills TX, LLC, Houston, TX (US)

(72) Inventors: Leann SALMONS, Houston, TX (US); Gretchen Glick-Mazziotti, Houston, TX (US)

(21) Appl. No.: 17/177,658

(22) Filed: Feb. 17, 2021

Related U.S. Application Data
(60) Provisional application No. 62/978,191, filed on Feb. 18, 2020.

(51) Int. Cl. A61K 35/747; A61K 9/00; A61K 31/05; A61K 31/352; A61K 47/44; A61K 35/745

(52) U.S. Cl. CPC A61K 35/747; A61K 9/0031; A61K 9/0034; A61K 35/745; A61K 31/352; A61K 47/44; A61K 31/05

(57) ABSTRACT

Cannabinoids and probiotics formulated into a suppository and used to treat urogenital, gastrointestinal or aural infections and/or inflammation by local use of the suppository for a period of time are described.

### PRIOR RELATED APPLICATIONS

This application claims priority to U.S. Ser. No. 62/978,191, filed Feb. 18, 2020, incorporated by reference in its entirety for all purposes.

### FEDERALLY SPONSORED RESEARCH STATEMENT

Not applicable.

### FIELD OF THE DISCLOSURE

The disclosure generally relates to novel combinations of cannabinoids and probiotics in suppository form and their uses in treating chronic diseases and conditions, including, but not limited to, urogenital (UG), gastrointestinal (GI), mental and psychologic disorders.

### BACKGROUND OF THE DISCLOSURE

Probiotics: The ‘microbiome’ is the genetic material of all the microbes—bacteria, fungi, protozoa and viruses—that live on and inside the human body. The number of genes in all the microbes in one person's microbiome is estimated at 200 times the number of genes in the human genome, and the microbiome may weigh as much as five pounds. These microbes help digest our food, regulate our immune system, protect against other bacteria that cause disease, and produce vitamins, including B vitamins (B12 thiamine and riboflavin) and Vitamin K, and many more important roles are sure to be discovered.

The Human Microbiome Project (HMP) was a United States National Institutes of Health (NIH) research initiative to improve understanding of the microbial flora involved in human health and disease. Launched in 2007, the first phase (HMP1) focused on identifying and characterizing human microbial flora. The second phase, known as the Integrative Human Microbiome Project (iHMP), launched in 2014 to characterize the microbiome and elucidate the roles of the microbes in health and disease states. By now, the complete genome of more than 2000 microbes in the microbiome have been sequenced, and work is ongoing to sequence, align and curate many more.

However, millennia before the HMP initiative, women around the world appreciated the value of vaginal flora in maintaining a healthy and comfortable vaginal environment. Thus, women have long treated, e.g., yeast infections, by supplementing with Lactobacillus cultures, both orally and locally. Thus, there is a long history of local application of probiotics to the vagina. In addition, probiotics have been applied rectally to the GI tract to aid in immunological function, digestion, protect against pathogenic bacteria such as Salmonella typhirnuriurn, Helicobacter pylori, and Escherichia coli, improve lactose intolerance, decrease cholesterol levels, treat Crohn's disease, ulcerative colitis, irritable bowel syndrome (“IBS”), and replenish intestinal flora after antibiotic therapy to prevent antibiotic-induced diarrhea. Indeed, the global market for probiotics reached a value of about 49.4 billion U.S. dollars in 2018.

Given the enormous market, it is not surprising that there are a great many patent relating to probiotics to treat GI and UG pathologies. For example, U.S. Pat. No. 6,180,100, teaches the use of Lactobacillus casei, L. acidophilus, L. plantarum, and L. jensenii for treatment of urinary tract infections. U.S. Pat. Nos. 5,176,911 and 6,277,370 use Lactobacilli for treatment of vaginal infections. U.S. Pat. No. 8,846,029 uses L. plantarum cells of strain 299 or 299 to treat inflammatory bowel disease (IBD) in the form of ulcerative colitis or Crohn's disease. U.S. Pat. No. 9,050,358 teaches treatment of IBS, diarrhea, alternating constipation/diarrhea, flatulence, or a constipation with viable non-pathogenic Clostridia spores and viable non-pathogenic Collinsella, wherein the non-pathogenic Clostridia is selected from the group consisting of Clostridium bifermentans, C. butyricum, C. difficile, C. ramosumn, or C. innocuum.

Cannabinoids: Endocannabinoids are produced naturally in humans and animals, phytocannabinoids in cannabis and some other plants; and synthetic cannabinoids are chemically manufactured. Phytocannabinoids are the naturally occurring molecules found in cannabis that give this herb its various properties. Humans have endocannabinoid receptors throughout the CNS and organ systems, which allows cannabis and the endocannabinoids to act on the human body. (FIG. 1). For the purposes herein, the term “cannabinoids” is intended to include the phytocannabinoids (e.g., Table 1), the endocannabinoids (e.g., arachidonoyl-ethanolamide (AEA) and 2-arachidonoyl-glycerol (2-AG)), as well as synthetic cannabinoids (e.g., AM 1241; JWH 133; GW 405833; JWH 015; HU 308: L-759,633; L-759,656; SER 601; GP 1a; GP 2a; CB 65; HU 210; CP 55,940; WIN 55, 212-2) and novel CB1, CB2 agonists.

Δ9-tetrahydrocannabinol (“THC”), is primary psychoactive compound of cannabis, but there are many other active cannabinoids in these plants. Cannabidiol (“CBD”), making up to 40% extracts of plant resin, has varied medical benefits, and at least 85 different cannabinoids have been isolated from cannabis (see e.g., Table 1). CBD has a greater affinity for CB2 than CB1 receptors and acts as serotonin (5-HT1A) receptor agonist, which may explain its antidepressant, anxiolytic, and neuro-protective effects. CBD also modulates opioid receptors involved with pain perception. CBD is not psychoactive and relieves convulsion (seizures), inflammation, anxiety, and nausea. It has been found to prevent short-term memory loss from THC. Antipsychotic effects of cannabidiol represents potential treatment of schizophrenia. See FIG. 2.

CBD is considered to have a wide scope of medical applications. An oral CBD formulation (Epidiolex®), is a liquid formulation of a CBD that received orphan drug status in the US for Dravet syndrome—an intractable seizure disorder (previously Severe Myoclonic Epilepsy of Infancy (SMEI)), Lennox-Gastaut syndrome, and tuberous sclerosis complex. Nabiximols (Sativex) is an aerosolized oral mist of CBD and THC approved in Canada for multiple sclerosis pain.

CBD has also been found safe and well tolerated as treatment for schizophrenia. A double-blind trial compared cannabidiol to atypical antipsychotic Amisuipride in acute paranoid schizophrenia. Both treatments showed significant decrease in psychotic symptoms, but cannabidiol had fewer side effects. Studies also show cannabidiol decreased symptoms of social anxiety and isolation. Cannabidiol has demonstrated antidepressant-like effects in animal models of depression, and is gaining notoriety as a potential treatment for a variety of mood disorders, especially generalized anxiety.

Cannabinoids are also known for their powerful anti-inflammatory effects. U.S. Pat. No. 6,410,588 for example, the use of cannabinoids to treat chronic diseases such as rheumatoid arthritis, glaucoma, macular degeneration, Crohn's Disease, sarcoidosis, asthma, Parkinson's Disease, Alzheimer's disease, ALS multiple sclerosis, muscular dystrophy, psoriasis, ulcerative colitis, osteoarthritis or spondyloarthropathy (erg. ankylosing spondylitis). WO2009043836 uses cannabidiol and denbinobin to treat GI inflammation and cancer. U.S. pat. No. 9,421,187 uses the phytocannabinoids tetrahydrocannabivarin (THCV), cannabigerol (CBG), cannabichromene (CBC), and cannabidivarin (CBDV), for use in the treatment of intestinal inflammatory diseases.

Some of the cannabinoids may also have anti-tumorigenic effects. US20160136127 and US20110086113, for example, teach the use of tetrahydrocannabinol (THC) and/or cannabidiol (CBD) for treating glioma.

Although both probiotics and cannabinoids are separately known for the prophylaxis and treatment of disease, they have not heretofore been combined. Although data is not yet available, we predict that the combination will provide synergistic effects, because the two active ingredients have differing mechanisms of action.

In addition, adequate delivery of either probiotics or cannabinoids has been an elusive goal. Many systems have been developed for delivery to the GI and UG systems, and commercial formulations consist mainly of food-based products, many of which use probiotic bacteria in their production with others having added these bacteria as an adjunctive health benefit of ingesting the product. These products account for about 90% of probiotic formulations and with the large amount of research into improvement of commercial food-based products for delivery of functional probiotic bacteria, their ability to act as probiotic delivery systems cannot be ignored. Cannabinoids are also often oral or inhaled, but inhalation methods have significant health risks. Dermal formulations are available, but bioavailability of cannabinoids appears to be limited (FIG. 3). There is also some use of suppositories, but these are a minute share of the market, as compared with foods, drinks, and pills. Thus, the targeted local delivery of probiotics and cannabinoids, by e.g., suppositories, remains under-researched and under-developed, with considerable room for improvement.

This invention relates to improved suppository formulations for local treatment of infectious and inflammatory diseases. The suppositories contain a novel combination of both probiotics and cannabinoids, such as CBD, thus assisting in rejuvenating the native flora and simultaneously decreasing inflammation, among other potential benefits.

### SUMMARY OF THE DISCLOSURE

This disclosure provides several suppository formulations for regional delivery of combined probiotics and cannabinoids, together with one or more optional secondary active ingredients and/or excipients, to be inserted in the vagina, urethra, rectum, ear canal, and the like. The probiotics will function to restore the normal microbiome, which can be altered by changes in diet, pH, sexually transmitted infections, antibiotic treatment, and the like. The cannabinoids have powerful anti-inflammatory effect, and thus provides both relief from burning and/or itching, as well as shrinking inflamed tissues and calming the cytokine cascade. The two together are predicted to have powerful synergistic effects on the UG, GI and aural systems.

The suppositories are particularly advantageous because they bypass the gastro-intestinal tract and deliver the actives directly to the lower gastro-intestinal tract, urethra, vagina and/or ear. The suppositories will dissolve/melt to release probiotic and cannabinoid contained therein, providing direct local relief from symptoms and in some cases also treating the underlying pathology.

Vaginal suppositories, for example, can be used for treatment and/or prophylaxis of bacterial vaginosis, viral vaginosis, candidiasis, in particular vulvovaginal candidiasis, bacterial vaginosis (BY), sexually transmitted diseases, such as HIV, gonorrhea, herpes, venereal ulcer, and chlamydia infection, infections endangering the fetus in pregnant women, preterm labor, urinary tract infection and pelvic floor dysfunction, with the proviso that Lactobacillus plantarum 299v, deposition number DSM 9843, is currently only known for urinary tract infection.

The suppositories can also be used to treat, e.g., all kind of acute and chronic inflammations and bacterial infections, especially of the gastrointestinal, vaginal or urethral tract. For example, the suppository can be used to restore the mucosal flora and to treat and/or prevent inflammatory bowel diseases, like colitis ulcerosa, necrotizing enterocolitis (NEC), or Morbus Crohn—especially as recrudescence prophylaxis—diarrhea, constipation (obstipation), cystitis, colitis, allergy, or neurodermatitis, and treat urogenital infections including, but not limited to, prostatitis.

Preferred probiotics for vaginal use include, Lactobacilli: acidophilus, brevis, bulgaricus, plantarum, rhamnosus, gasseri, casei, salivarius, paracasei, fermentum, salivarius, reuteri, helveticus, lactis, cremoris, kefiri, parakefir, faecium, crispatus, delbrueckii, and Lactococcus: lactus.

Preferred rectal probiotics include Lactobacilli: acidophilus, brevis, bulgaricus, plantarum, rhamnosus, gasseri, casei, salivarius, paracasei, rueten, fermentum, breve, animalis, sreuteri, helvecticus, kefiranofaciens, kefigranum, lactic, cremoirs, themophilus, kefiri, parakefir, lactisbiovardiacetylactis, faceium, crispatus, iners, jensienii, sporagenus, delbrueckii; and Bifidobacteria, breve, bifidum, infantis, lacti longum.

Preferred aural probiotics include Lactobacilli: acidophilus, and plantarum.

Preferred urethral probiotics include Lactobacilli: acidophilus, bulgaricus, plantarum, rhamnosus, gasseri, casei, helveticus, lactis, cremoris, kefiri, parakefir, faecium, crispatus, delbrueckii, and Lactococcus lactus.

Cannabinoids may include any of the cannabinoids listed in Table 1, but CBD and CBG may be preferred, as well as other CB2 agonists.

Preferred cannabinoids, probiotics and other ingredients are then inserted for novel delivery (vaginal, rectal, aural, urethral), thereby bypassing the gastrointestinal tract and are not subjected to metabolization in the liver (11-hydroxy-THC) as in oral consumption; but deliver the combination regionally. Therefore, the preferred cannabinoid formulations have improved bioavailability. Also preferred are formulations with reduced psychoactive symptoms for THC (and its derivatives).

As used herein, “probiotic” refers to non-pathogenic microorganisms that have beneficial uses in the human body. They are live microorganisms that may be able to help prevent and treat some illnesses. Promoting a healthy digestive tract and vagina, as well as a healthy immune system are their most widely studied benefits at this time. These are also commonly known as friendly, good, or healthy bacteria.

As used herein, a “cannabinoid” includes any of the phytocannabinoids endocannabinoids or synthetic cannabinoids. Preferred are the various naturally-occurring, biologically active, chemical constituents (such as cannabidiol or cannabinol) of hemp or cannabis including some (such as THC) that possess psychoactive properties, plus any synthetic mimetics. All classes derive from cannabigerol-type (CBG) compounds and differ mainly in the way this precursor is cyclized. The classical cannabinoids are derived from their respective 2-carboxylic acids (2-COOH) by decarboxylation (catalyzed by heat, light, or alkaline conditions).

As used herein, “suppository” is a solid medical preparation in a roughly conical, spherical, tear drop, cylindrical or other convenient shape, designed to be inserted into the rectum, vagina or other orifice to melt or dissolve, thus delivering pharmaceutical agents locally. In some cases, the suppository is large and firm enough to be inserted manually, in others an applicator is used, such as with urethral suppositories.

As used herein, an “effective amount” is an amount needed to treat disease or alleviate a symptom in a majority of individuals of a given body weight, and specifically excludes the minute amounts that are used in so-called “homeopathic” remedies.

As used herein, “supplement” is a product taken orally that contains one or more ingredients (such as vitamins or amino acids) that are intended to supplement one's diet and are not considered food or drugs under the US regulatory scheme.

As used herein, the term “excipient” means the substances used to formulate active pharmaceutical ingredients (API) into pharmaceutical formulations. Excipients (e.g., mannitol, Captisol®, lactose, starch, magnesium stearate, sodium saccharine, talcum, cellulose, sodium crosscarmellose, glucose, gelatin, sucrose, magnesium carbonate, C10 fatty acids, and the like) are an integral part of pharmaceutical development and help to achieve the desired product profile including but not limited to an aid in manufacturing, modify a drug's stability, and efficacy. Pharmaceutically acceptable excipients are non-toxic and do not adversely affect the therapeutic benefit of at least one chemical entity described herein. Such excipient may be any solid, liquid, semi-solid or, in the case of an aerosol composition, gaseous excipient that is generally available to one of skill in the art.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication.

As used herein, the term “pharmaceutical composition” or “pharmaceutical formulation” describes one or more active ingredients and one or more pharmaceutically acceptable excipients.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the invention, such as instructions for use, buffers, flavorants, colorants, melt/dissolution delay coatings, and the like.

The following abbreviations are used herein:

| ABBREVIATION | TERM |
|---|---|
| 2-AG | 2-arachidonolglycerol |
| 5-HT | 5-hydroxytryptamine receptors or |
| | serotonin receptor |
| AEA | Anandamide |
| BK | Bradykinin |
| BV | Bacterial vaginosis |
| CB1 | Cannabinoid receptor 1 |
| CB2 | Cannabinoid receptor 2 |
| CBC | Cannabichromene |
| CBCA | Cannabichromenenic acid |
| CBCV | Cannabichromevarin |
| CBCVA | Cannabichromevarinic acid |
| CBD | Cannabidiol |
| CBDA | Cannabidiolic acid |
| CBDV | Cannabidivarin |
| CBDVA | Cannabidivarinic acid |
| CBG | Cannabigerol |
| CBGA | Cannabigerolic acid |
| CBGV | Cannabigerivarin |
| CBGVA | Cannabigerovarinic acid |
| CGRP | calcitonin gene-related peptide |
| Et | Ethanolamine |
| FAAH | Fatty acid amide hydrolase |
| GI | Gastrointestinal |
| IBD | inflammatory bowel disease |
| IBS | inflammatory bowel syndrome |
| iR | ionotropic receptor |
| MCT | Medium chain triglycerides |
| mR | metabotropic receptor |
| NT | Neurotransmitter |
| PP | Prostaglandins |
| SP | Substance P |
| THC | Δ9-tetrahydrocannabinol |
| THCA | Δ9-tetrahydrocannabinolic acid |
| THCV | Tetrahydrocannabivarin |
| THCVA | Tetrahydrocanabivarinic acid |
| UG | Urogenital |
| NOS | Not otherwise specified |

### BRIEF DESCRIPTION OF THE DRAWINGS

FIG. 1. Human endocannabinoid system. Showing CB1 and CB2 receptors throughout the human body.

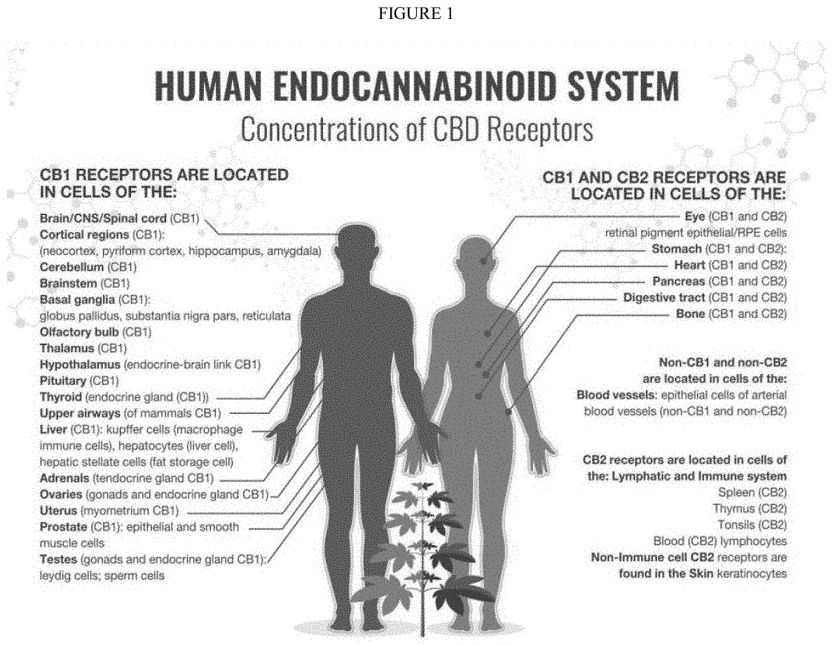

FIG. 2. Simplified scheme representing the pathogenesis of pain following inflammatory disease or nociceptive stimulus, the cytokines involved in the process, the descending supraspinal modulation and the relive neurotransmitters and endocannabinoid retrograde signaling mediated synaptic transmission. Endocannabinoids are produced from postsynaptic terminals upon neuronal activation. Natural and synthetic cannabinoids act like the two major endocannabinoids shown in the scheme: 2-arachidonolglycerol (2-AG) and anandamide (AEA). Endocannabinoids readily cross the membrane and travel in a retrograde fashion to activate CBI located in the presynaptic terminals. Activated CB1 will then inhibit neurotransmitter (NT) release through the suppression of calcium influx. NT can bind to ionotropic (iR) or metabotropic (mR) receptors. 2-AG is also able to activate CB1 located in astrocytes. Although endocannabinoid retrograde signaling is mainly mediated by 2-AG, AEA can activate presynaptic CB1 as well. Fatty acid amide hydrolase (FAAH) found in postsynaptic terminals is responsible for degrading AEA to AA and ethanolamine (Et). Inflammation lead to release of biochemical mediators (bradykinin (BK), serotonin (5-HT), prostaglandins (PG) etc.) and the up-regulation of pain mediator nerve growth factor (NGF). The substance P (SP) and calcitonin gene-related peptide (CGRP) vasoactive neuropeptides, released from sensory nerve, have also role in inflammation. The interaction with opioids, THC and nonsteroidal anti-inflammatory drugs are also represented.

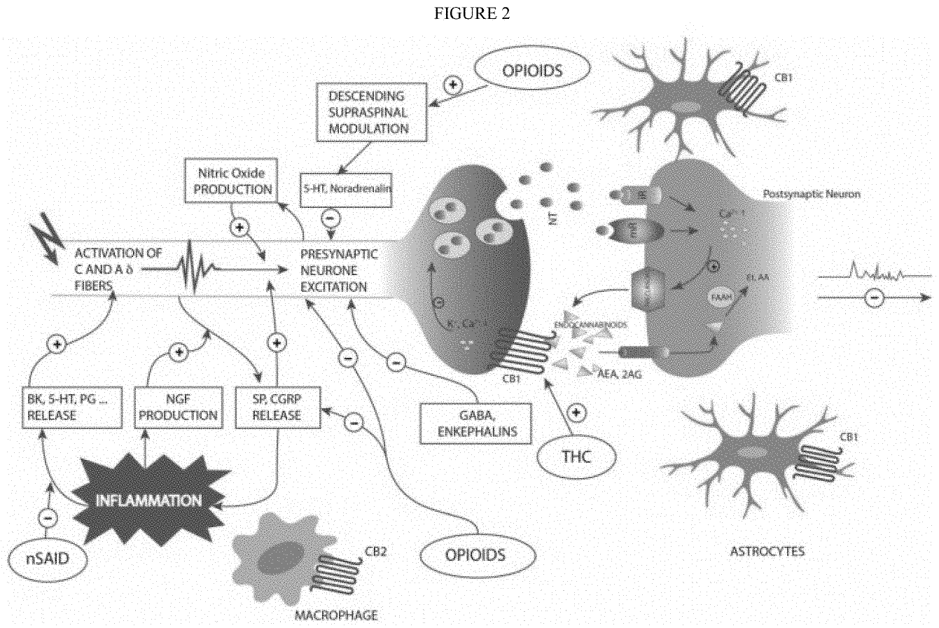

FIG. 3. Cannabinoid bioavailability.

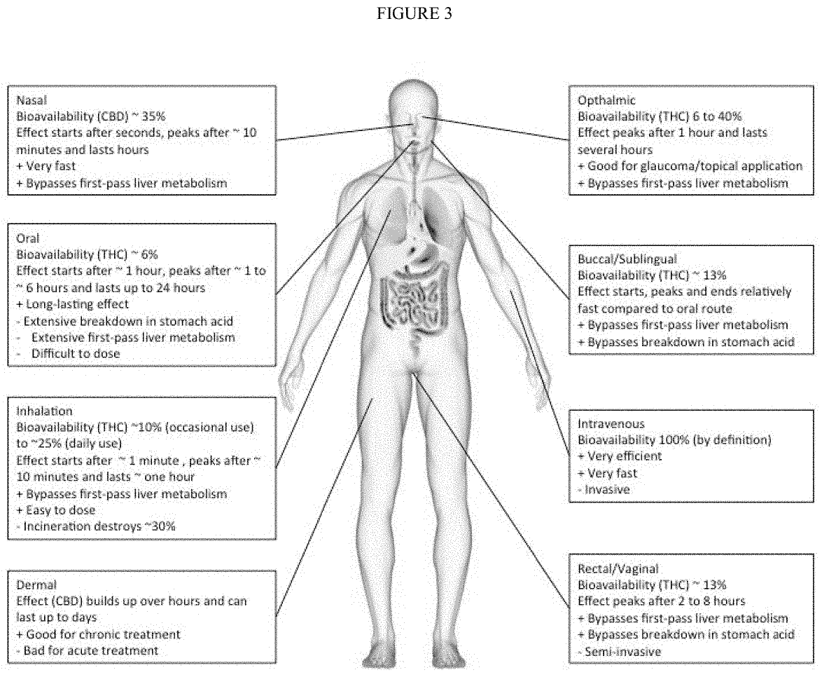

### DETAILED DESCRIPTION

The disclosure provides novel suppository formulations, such as those shown in the examples below, each formulation containing one or more probiotics and one or more cannabinoids in a suppository base.

Table 1 describes some of the most common cannabinoids that may be used in the invention, each of which may also be present in acid form (e.g., CBD and CBDA). The THC is preferably largely absent or only used in trace amounts (e.g., <0.3 mg/dose) but in some indications may be much higher, e.g., in a 1:1 ratio with CBD:THC or a 10:1: or 2:1, or 1:10 or 1:2 ratio. All cannabinoids combined are between <50-500 mg/dose, or between <225-450 or most preferred about 300 mg/dose, although as noted in some jurisdictions THC may only be present in trace amounts.

TABLE 1

Cannabinolds
Cannabichromene (CBC)
Cannabichromenic acid (CBCA)
Cannabichromevarin (CBCV)
Cannabichromevarinic acid (CBCVA)
Cannabicyclol (CBL)
Cannabidihexol (CBDH)
Cannabicyclolic acid (CBLA)
Cannabicyclovarin (CBLV)
Cannabidiol (CBD)
Cannabidiol monomethylether (CBDM)
Cannabidiolic acid (CBDA)
Cannabidiorcol (CBD-C1)
Cannabidivarin (CBDV)
Cannabidivarinic acid (CBDVA)
Cannabielsoic acid B (CBEA-B)
Cannabielsoin (CBE)
Cannabielsoin acid A (CBEA-A)
Cannabigerol (CBG)
Cannabigerol monomethylether (CBGM)
Cannabigerolic acid (CBGA)
Cannabigerolic acid monomethylether (CBGAM)
Cannabigerovarin (CBGV)
Cannabigerovarinic acid (CBGVA)
Cannabinodiol (CBND)
Cannabinodivarin (CBVD)
Cannabinol (CBN)
Cannabinol methylether (CBNM)
Cannabinol-C2 (CBN-C2)
Cannabinol-C4 (CBN-C4)
Cannabinolic acid (CBNA)
Cannabiorcool (CBN-C1)
Cannabivarin (CBV)
Cannabichromevarin (CBCV)
10-Ethoxy-9-hydroxy-delta-6a-tetrahydrocannabinol
8,9-Dihydroxy-delta-6a-tetrahydrocannabinol
Cannabitriol (CBT)
Cannabitriolvarin (CBTV)
Delta-8-tetrahydrocannabinol (Δ8-THC)
Delta-8-tetrahydrocannabinolic acid (Δ8-THCA)
Delta-9-tetrahydrocannabinol (THC)
Delta-9-tetrahydrocannabinol-C4 (THC-C4)
Delta-9-tetrahydrocannabinolic acid A (THCA-A)
Delta-9-tetrahydrocannabinolic acid B (THCA-B)
Delta-9-tetrahydrocannabinolic acid-C4 (THCA-C4)
Delta-9-tetrahydrocannabiorcol (THC-C1)
Delta-9-tetrahydrocannabiorcolic acid (THCA-C1)
Delta-9-tetrahydrocannabivarin (THCV)
Delta-9-tetrahydrocannabivarinic acid (THCVA)
10-Oxo-delta-6a-tetrahydrocannabinol (OTHC)
Cannabichromanon (CBCF)
Cannabifuran (CBF)
Cannabiglendol
Cannabiripsol (CBR)
Cannbicitran (CBT)
Dehydrocannabifuran (DCBF)
Delta-9-cis-tetrahydrocannabinol (cis-THC)
Tryhydroxy-delta-9-tetrahydrocannabinol (triOH-THC)
3,4,5,6-Tetrahydro-7-hydroxy-alpha-alpha-2-trimethyl-9-n-propyl-
2,6-methano-2H-1-benzoxocin-5-methanol, (OH-iso-HHCV)

The cannabinoid may be synthetic and partially purified or be pharmaceutical grade, or may be an extract from a Cannabis plant, as the combined formulations from the native plants are preferred in some cases. Synthetic mimetics and CB2 agonists may also be used.

Table 2 describes some of the probiotics that may be used in the invention. As a general proposition, the total pharmaceutically effective amounts of probiotics administered per dose will be in the range of 1 million to 1000 billion colony forming units (CFUs), or 5 billion to 100 billion, or 6 billion to 90 billion CFUs in total per dose. The quantity may vary according to the intended region of treatment, e.g., the vagina allowing higher doses than the ear.

TABLE 2
Exemplary Probiotics

| | |
|---|---|
| Atopobium | Vaginae |
| Bacillus | Subtillis |
| Bacillus | Cereus |
| Bacillus | Clausii |
| Bacillus | Coagulans |
| Bacillus | Indicus |
| Bifidobacteria | Breve |
| Bifidobacteria | Bifidum |
| Bifidobacteria | Infantis |
| Bifidobacteria | Lactis |
| Bifidobacteria | Longum |
| Brettanomyces | Anomalus |
| Brettanomyces | Hansenii |
| Debaryomyces | Hansenii |
| Enterococcus | Faecuim |
| Gardnerella | Vaginalis |
| Kluyveromyces | marxianus |
| Lactobacilli | Acidophilus |
| Lactobacilli | brevis |
| Lactobacilli | bulgaricus |
| Lactobacilli | plantarum |
| Lactobacilli | rhamnosus |
| Lactobacilli | gasseri |
| Lactobacilli | casei |
| Lactobacilli | salivarius |
| Lactobacilli | paracasei |
| Lactobacilli | rueten |
| Lactobacilli | fermentum |
| Lactobacilli | breve |
| Lactobacilli | animalis |
| Lactobacilli | salivarius |
| Lactobacilli | reuteri |
| Lactobacilli | helveticus |
| Lactobacilli | kefiranofaciens |
| Lactobacilli | kefirgranum |
| Lactobacilli | lactis |
| Lactobacilli | cremoris |
| Lactobacilli | thermophilus |
| Lactobacilli | kefiri |
| Lactobacilli | parakefir |
| Lactobacilli | lactis biovar diacetylactis |
| Lactobacilli | faecium |
| Lactobacilli | crispatus |
| Lactobacilli | iners |
| Lactobacilli | jensenii |
| Lactobacilli | sporagenus |
| Lactobacilli | delbrueckii |
| Lactococcus | lactis biovar diacetyltics |
| Lactococcus | lactis |
| Lactococcus | cremoris |
| Leuconostoc | lactis |
| Leuconostoc | mesenteroides |
| Leuconostoc | cremoris |
| Leuconostoc | dextranicum |
| Saccharomyces | unisporus |
| Saccharomyces | turicensis |
| Saccharomyces | exiguus |
| Saccharomyces | boulardii |
| Saccharomyces | cerevisiae |
| Streptococcus | salvarius |
| Streptococcus | thermophilus |
| Torulaspora | Delbrueckii |

Other active ingredients that may advantageously be combined with cannabinoids and probiotics include those in Table 3:

TABLE 3
Additional ingredients

| | |
|---|---|
| Mushrooms | Psilocybin (P. azurescens, P. semilanceata, P. |
| | cyanescens, P. subaeruginosa, P. bohemica, P. |
| | cubensis, P. cynanofibrillosa) 5-10 mg 10-25 mg, |
| | 30-45+ mg |
| & root network | Cordyceps militaris & sinensis (mycelium) |
| & adaptogens | Organic reishi (ganoderma lucidum) |
| | Organic lions mane (hericiumerinaceus) |
| | Organic shitake (lentinus edodes/mycelium) |
| | Organic Poria |
| | Organic Maitake |
| | Organic Himematsutake (Royal Sun Agaricus) |
| | Oyster Mushrooms |
| | Porcini |
| | White Button Mushrooms |
| | Shimeji |
| | Chanterelle |
| | Black truffle |
| | Organic Turkey Tail |
| | Ashwagandha |
| | Rhodiola |
| | Tulsi |
| | Chaga (lnonotus obliquus) |
| | MDMA_Methylenedioxy methamphetamine |
| | Burdock root |
| | Saint Johns Wort |
| | Sepia |
| | Natrum mur |
| | Mercurius solubilis (Merc sol) |
| | Kreosote |
| | Medorrhium |
| | Pulsatilla |
| | Alumina |
| | Sanicula |
| | Cantharis |
| | Calcarean, carbonica |
| | Boric acid |
| | Suphur |
| | Ceramides |
| | Bacopa Monnieri |
| | Vitamin C (ascorbic acid) Ester of Vitamin C |
| | Estriol (E3), |
| | Estradiol, estrone |
| | (Oestriol, Theelol, trihydoxyestrin, |
| | trihydr9xy9estr8hkn16a-hydroxyestradiol, Estra- |
| | 1,3,5,(10),-triene-3, 16a, 17beta-triol. |
| | Ginkgo Biloba |
| | Curcumin |
| | Piper Nigrum (black pepper) |
| | All B vitamins (B-1, B-2, B-3, B-5, B-9); vitamin D-3, |
| | Zinc, Magnesium, sodium |
| | Chicory root |
| | Aloe vera |
| | Fulvic Acid |
| | Microcrystalline cellulose |
| | Colostrum (whole or freeze dried-Bovinae) |
| | Lysozyme hemicellulase |
| | clove extract |
| | Antifungal agents, such as Clotrimazole, Nystatin, |
| | Ciclopirox, and Fluconazole. |
| | Testosterone |
| | Hops |
| | Echinacea |
| | Selenium |
| | L-Arginine |
| | Ginseng |
| | Pomegranate extract |
| | Icariin (horny goat weed) |
| | panax |
| | Propionyl- L-Carnitine |
| | L-Citrulline |
| | Rhodiola rosea (golden root) |
| | Mastic gum (yohimbine hydrochloride) |
| | Indian Ginseng |
| | Bromelain |
| | Nitric oxide |
| | N-acetyl cysteine (NAC) |
| | d-mannose |
| Preferred fats: | MCT oil, olive oil, coconut oil, hemp seed oil, |
| | canola oil, sunflower oil, avocado oil, safflower |
| | oil, grape seed oil, peanut oil, maize, walnut oil, |
| | sesame oil, ghee, palm oil, castor oil, Theobroma |
| | oil. |
| Other (binder): | Glycerol-gelatin, macrogol |
| Terpenes | α-Humulene |
| | myrcene |
| | α-pinene, β-Caryophyllene |
| | Other Cytokines, Defensins |
| | Geraniol |
| | Limonene |
| | Linalool |
| | Ocimene |
| | Trans-β-ocimene |
| | terpineol |
| | Valencene |
| | Trans-nerolidol |
| | Bisabolol |
| | Delta 3 carene |
| | Eucalyptol |
| | Camphene |
| | Borneol |
| | Terpinene |
| | Guaiol |
| | Terpinolene |
| | Phellandrene |
| | Carene |
| | Fenchol |
| | Bisabolol |
| | Phytol |
| | Camphor |
| | Sabinene |
| | Isoborneol |
| | Menthol |
| | Nerolidol |
| | Cedrene |
| | Isopulegol |
| | Cymene |
| | Pulegone |
| Flavinoids | beta-sitosterol |
| | quercetin |
| | apigenin |
| Cannaflavines | Cannaflavin-a |
| Flavinoids | beta-sitosterol |
| | Quercetin |
| Miscellaneous | Black cohosh |
| | Denbinobin, 1,4 phenanthrenequinone |
| | Coprococcus Clostridia Eutaetus |
| | Dialister |

Cannabinoids are highly lipophilic molecules with very low aqueous solubility and are susceptible to degradation, especially in solution, via the action of light and temperature as well as via auto-oxidation. Thus, preferred bases for the suppository are lipophilic. Micellar formulations may also be used.

While the base for the suppositories can include any known or to be developed formulation, the base is preferably any hard fat that is solid at room temperature, and melts at 37° C. Mixtures of oils, surfactants, solvents and co-solvents/surfactants can also be used.

Generally, the fat (or fat mixture) is melted, mixed evenly with the active ingredients, poured into a mold to harden, then individually packaged, e.g., in foil. Preferred hard fats for the base include: coconut oil, ghee, palm oil, Theobroma oil (cocobutter), and others.

The base can also be mineral oil plus a thickener. Thickeners can be completely natural, like waxes, and also synthetic or semi-synthetic polymers and the like, including polysaccharides, proteins, alcohols, silicones or waxes. Suitable thickeners may include bees wax, candelilla wax, carnauba wax, paraffin, Ozokerite wax, cetyl alcohol, corn starch, glyceryl stearate, guar gum, gum Arabic, xanthan gum, lanolins, microcrystalline wax, acrylate polymers, polyalphaolefins, HE-Cellulose, PEG-150 Distearate, sorbitol, stearic acid, stearyl palmitate, Poloxamer 407, and the like.

A preferred base may include a proprietary blend of low density polyethylene known as PCCA Plasticized™ (PCCA US, TX, Cat. No. 30-3211). Even more preferred is a blend of 10-25%, 15-21% or 18% of United States Pharmacopoeia (USP) or National Formulary (NF) paraffin brought to 100% (ww) with USP or NF mineral oil.

Liquid fats can also be mixed in with the hard fats as long as the suppositories remain solid at room temperatures. Liquid fats include MCT oil, olive oil, hemp seed oil, canola oil, sunflower oil, avocado oil, safflower oil, grape seed oil, peanut oil, maize, walnut oil, sesame oil, castor oil, and others.

In some embodiments the suppository may be coated with a material to delay is melting/dissolution. In other embodiments, the ingredients may be sequestered in different portions of the suppository. For example, we may find that probiotic viability is improved if not intimately admixed with the cannabinoids. Thus, the probiotics may be centralized and the cannabinoids on the exterior or vice versa. Thus, although the various ingredients are dispersed in the base, they may be dispersed in different portions of the base. Further, where they are sequestered in whole or part, the base can then be optimized for the delivery of the different components.

We anticipated performing viability studies to ensure that the probiotics are viable even when formulated into a suppository. The probiotics will be formulated as above, the suppository melted, dilutions performed and plated on agar plates and colonies counted. It may be necessary to adjust the formulae for probiotic health. However, suppository formulations of probiotics are known, and no significant difficulties are expected, although it may be beneficial to add a nutrient source for the probiotics as well as pH controlling agents (e.g., buffers). Obviously, the pH may be different for a vaginal suppository than others.

Other ingredients that can be use in the suppository base include, e.g., cocoa butter or a similar substitute, polyethylene glycol, hydrogels, and glycerinated gelatin. The type of material used depends on the type of suppository, the type of drug, and the conditions in which the suppository will be stored.

The term “hard fat” as used herein means a mixture of the monoglyceride, diglyceride and triglyceride of usually straight-chain saturated fatty acids containing 8 to 18 carbon atoms that are solid at room temperature and examples of such mixture are mentioned in the literature. Preferred fats are C8-C12.

Preferably, the hard fat has a hydroxyl value of 50 or less. The hard fats having a hydroxyl value not more than 50 are commercially available, for example under the trade marks, Witepsol H-35, H-5, H-15 and W-35 by Dynamit Nobel Co. and Nissan Pharmasol B-115 by Nippon Oil & Fats Co., Ltd. Any suppository manufactured using a hard fat with a hydroxyl value in excess of 50 shows a poor shelf life and tends to become colored during storage and transit.

In some embodiments for vaginal use, starch and/or estriol can be added to the suppository, each having potential growth effects on the probiotic by providing energy from the breakdown of the starch, or from the glycogen secreted in response to the estriol.

The invention includes any one or more of the following embodiments, in any combination(s) thereof:

A suppository formulation for the local treatment of infection or inflammation or both, said formulation comprising a base that is a solid at room temperature and that liquifies or dissolves at about 37° C. in a human body; one or more probiotics dispersed in said base; one or more cannabinoids dispersed in said base; and said formulation being in the format of a solid suppository.

Any formulation herein, said cannabinoid being at <50-500 mg per dose, or about 200, 300, or 400 mg/dose.

Any formulation herein, said probiotic being at 1 million to 1000 billion CFUs per dose, or 1 billion to 100 billion CFUs, or about 10-50 billion CFUs.

Any formulation herein, said cannabinoid being at CBD and THC at about a 1 to 1 ratio CBD:THC or as much as 10:1 or 1:10. In other embodiments, the THC can be present only in trace amounts, depending on both indication and local laws.

Any formulation herein, said base being selected from coco butter or coconut oil.

Any formulation herein, for use in the vagina, said probiotic selected from the group consisting of Lactobacillus, Bifidobacteria, and Lactococcus and said cannabinoid selected from the group consisting of CBGA, CBG, CBD, THCA, THC, THCVA, and THCV.

Any formulation herein, for use in the vagina, said probiotic selected from the group consisting of Lactobacilli: acidophilus, brevis, bulgaricus, plantarum, rhainnosus, gasseri, casei, salivarius, paracasei, fermentwn, salivarius, reuteri, helveticus, lactis, creinoris, kefiri, parakefir, faecium, crispatus, delbrueckii, and Lactococcus lactus, and said cannabinoid selected from the group consisting of CBGA, CBG, CBD, THCA, THC, THCVA and THCV.

Any formulation herein, for use in the rectum, said probiotic selected from the group consisting of Lactobacillus, Lactococcus, and Bifidobacteria, and said cannabinoid selected from the group consisting of CBGA, CBG, CBD, TCHA, THC, THCVA, and THCV.

Any formulation herein for use in the rectum, said probiotic selected from the group consisting of Lactobacilli: acidophilus, brevis, bulgaricus, plantaruin, rhamnosus, gasseri , casei, salivarius, paracasei, rueten, fermentum, breve, animalis, sreuteri, helvecticus, kefiranofaciens, keligranum, lactis, cremoirs, themophilus, kefiri, parakefir, lactisbiovardiacetylactis, faceium, crispatus, iners, jensienii, sporagenus, and delbrueckii and Bifidobacteria, breve, bifidum, infantis, lactis, and longum, and said cannabinoid selected from the group consisting of: CBGA, CBG, THCA, THC, CBDA, CBD, CBCA, CBC, CBVA, THCV, CBDVA, CBDV, CBN, CBNA, and CBNA.

A formulation being i) CBGA, CBG, THCA, THC, CBDA-CBD, ii) Lactobacilli: acidophilus, plantarum, casei, breve, and brevis, iii) Bifidobacteria: breve, bifidum, and lactis, and iv) boric acid.

A formulation being i) Lactobacilli: acidophilus, brevis, bulgaricus, plantarum, rhamnosus, gasseri, casei, salivarius, paracasei, fermentum, salivarius, reuteri, helveticus, lactis, cremoris, kefiri, parakefir, faecium, crispatus, and delbrueckii, ii) Lactococcus lactus, and iii) CBGA, CBG, CBD, THCA, THC, THCVA and THCV.

A formulation being i) Lactobacilli: acidophilus, brevis, bulgaricus, plantarum, rhamnosus, gasseri, casei, salivarius, paracasei, rueten, fermentum, breve, animalis, sreuteri, helvecticus, kefiranofaciens, kefigranum, lactis, cremoirs, themophilus, kefiri, parakefir, lactisbiovardiacetylactis, faceium, crispatus, iners, jensienii, sporagenus, and delbrueckii; ii) Bifidobacteria, breve, bifidum, infantis, lactis, and longum, and iii) said cannabinoid selected from the group consisting of: CBGA, CBG, THCA, THC, CBDA, CBD, CBCA, CBC, CBVA, THCV, CBDVA, CBDV, CBCV, CBN, and CBNA.

A method of treating a vaginal infection, disease or inflammation, said comprising administering a vaginal suppository to a vagina having an infection or inflammation at least daily for at least three days, or 7-21 days.

A method of treating a gastrointestinal disease or inflammation said comprising administering a suppository as herein described having the suppository formulation of claim 1 or 8 to a rectum having a gastrointestinal disease or inflammation at least daily for at least three days, or 7-21 days.

A method of treating an aural or neural condition, said comprising administering a suppository as herein described to an ear at least daily for at least three days, or 7-21 days.

A method of treating a urogenital infection, disease or inflammation, said comprising administering a suppository as herein described to a urethra having an infection or inflammation at least daily for at least three days or 7-21 days.

The present invention is exemplified with respect to vaginal and rectal suppositories, especially for treating BV and diarrhea. However, this is exemplary only, and the invention can be broadly applied to any inflammatory, autoimmune, infectious disease, and the like that is suitably treated with a suppository. The following examples are intended to be illustrative only, and not unduly limit the scope of the appended claims.

### Vaginal Formulations

Vaginal formulations can be used to treat any vaginal infection or imbalance or inflammation, including e.g., BV, STIs, and the like. All formulations herein are provided on a per dose basis and brought to a <1-8 gram suppository with e.g., coconut oil or C6: caproic acid or hexanoic acid, C8: caprylic acid or octanoic acid, C10: capric acid or decanoic acid and mixtures thereof.

TABLE 5
Formations 1-4 for vaginal use

| Ingredient | Function | F1 | F2 | F3 | F4 |
|---|---|---|---|---|---|
| CBGA/CBG, CBGVA, | Anti- | Trace - | 50-100 mg | 100- | 400 mg+ |
| CBGV, CBN, CBDA/CBD | inflammatory | <50 mg | | 400 mg | |
| Δ9-tetrahydrocannabinolic | Pain | Trace- | 50-100 mg | 100-400 mg | 400 mg+ |
| acid/Δ9- | management | <50 mg | | | |
| tetrahydrocannabinol | | | | | |
| Lactobacilli: acidophilus, | Micro-biome | <6 billion | <6 billion | <6 billion | <6 billion |
| brevis, bulgaricus, | support | CFUs, 6 | CFUs, 6 | CFUs, 6 | CFUs, 6 |
| plantarum, rhamnosus, | | billion- 90 | billion- 90 | billion- 90 | billion- 90 |
| gasseri, casei, salivarius, | | billion CFUs, | billion | billion | billion |
| paracasei, fermentum, | | 90 billion + | CFUs, 90 | CFUs, 90 | CFUs, 90 |
| salivarius, reuteri, | | CFUs. | billion + | billion + | billion + |
| helveticus, lactis, cremoris, | | | CFUs. | CFUs. | CFUs. |
| kefiri, parakefir, faecium, | | | | | |
| crispatus, delbrueckii, | | | | | |
| Lactococcus: lactus | | | | | |
| Diflucan/Nizoral, | Antifungal, local | <.1 or = | <.1 or = | <.1 or = | <.1 or = |
| Fluconazole, Clotrimazole | and systemic | 200 mg | 200+ mg | 200+ mg | 400+ mg |
| Boric acid (dominant form | Anti-itch | <or = 600 mg | < or = | < or = | < or = |
| of boron I plasma) | | | 600+ mg | 600+ mg | 600+ mg |
| Pulsatilla | Anti- | <.1 to 15+ g | <.1 to 15+ g | <.1 to 15+ g | <.1 to 15+ g |
| | inflammatory | | | | |
| Mannose (d-mannose)- | Prevention/ | < or =2 g | 1.5+ grams | 1.5+ grams | 1.5+ grams |
| prevention of recurrent | treating UTI | | | | |
| bacterial infections | | | | | |
| Bovine Colostrum, | antibacterial, | < or = | 500+ mg | 500+ mg | 500+ mg |
| containing Lactoferrin | support for | 500 mg | | | |
| Proteins | molecular | | | | |
| | innate | | | | |
| | immunity, that | | | | |
| | include | | | | |
| | cytokines, | | | | |
| | antimicrobial | | | | |
| | proteinis and | | | | |
| | peptides | | | | |
| Organic coco butter | binder | <1-8 grams | <1-8 grams | <1-8 grams | <1-8 grams |
| dimensions | Torpedo | <1 to 8 | <1 to 8 | <1 to 8 | <1 to 8 |
| | (shape) | grams and | grams and | grams and | grams and |
| | | 20 mm long | 20 mm long | 20 mm long | 20 mm long |

### Rectal Formulations

Rectal formulations can be used to treat any GI infection or imbalance or inflammation, including e.g., diarrhea, and the like.

TABLE 6
Formations 1-4 for rectal use

| Ingredient | Function | Amount F1 | Amount F2 | Amount F3 | Amount F4 |
|---|---|---|---|---|---|
| CBGA/CBG/THCA/TCH, | Anti | Trace - | 50-100 mg | 100- | 400 mg+ |
| THCVA/THCV | inflammatory, | <50 mg | | 400 mg | |
| Probiotics: Lactobacilli: | Microbiome | <6 billion | <6 billion | <6 billion | <6 billion |
| acidophilus, brevis, | support | CFUs, 6 | CFUs, 6 | CFUs, 6 | CFUs, 6 |
| bulgaricus, plantarum, | | billion- 90 | billion- 90 | billion- 90 | billion- 90 |
| rhamnosus, gasseri, casei, | | billion | billion | billion | billion |
| salivarius, paracasei, rueten, | | CFUs, 90 | CFUs, 90 | CFUs, 90 | CFUs, 90 |
| fermentum, breve, animalis, | | billion + | billion + | billion + | billion + |
| sreuteri, helvecticus, | | CFUs. | CFUs. | CFUs. | CFUs. |
| kefiranofaciens, kefigranum, | | | | | |
| lactis, cremoirs, themophilus, | | | | | |
| kefiri, parakefir, | | | | | |
| lactisbiovardiacetylactis, | | | | | |
| faceium, crispatus, iners, | | | | | |
| jensienii, sporagenus, | | | | | |
| delbrueckii; and | | | | | |
| Bifidobacteria, breve, | | | | | |
| bifidum, infantis, lactis, | | | | | |
| longum. | | | | | |
| Ashwagandha, rhodiola, tulsi, | Adaptogens, | <800 mg- | <800 mg- | <800 mg- | <800 mg- |
| chaga | traditional for | 800 mg+ | 800 mg+ | 800 mg+ | 800 mg+ |
| | anti-anxiety, | | | | |
| Bovine Colostrum, containing | Immune support | < or = | 500+ mg | 500+ mg | 500+ mg |
| Lactoferrin Proteins | and defensin | 500 mg | | | |
| | action | | | | |
| Organic coco butter | binder | <1-8 grams | <1-8 grams | <1-8 grams | <1-8 grams |
| dimensions | Torpedo | <1 to 8 | <1 to 8 | <1 to 8 | <1 to 8 |
| | (shape) | grams and | grams and | grams and | grams and |
| | | 20 mm long | 20 mm long | 20 mm long | 20 mm long |

### Urethral Formulations

Urethral formulations may be used to treat e.g., cervical cancer, Prostate cancer, BPH, neurogenic bladder, erectile dysfunction. Urethral and aural suppositories need to be much smaller than vaginal and rectal, so the size of the suppository will be adjusted accordingly, and suppository dosing may be more frequent to account for the dose reduction.

TABLE 7
Formations 1-4 for urethral use

| Ingredient | Function | Amount F1 | Amount F2 | Amount F3 | Amount F4 |
|---|---|---|---|---|---|
| CBGA/CBG/THCA/TCH, | (vasal dilation) | Trace - | 50-100 mg | 100-400 mg | 400 mg+ |
| THCVA/THCV | Pain, anti- | <50 mg | | | |
| | inflammatory | | | | |
| Boric acid | Anti-itch | < or = 600 mg | < or = | < or = | < or = |
| | | | 600+ mg | 600+ mg | 600+ mg |
| Quercetin, white button | Terpenes/ | < or = 2 g | 1.5 g+ | 1.5 g+ | 1.5 g+ |
| mushroom, d-mannose | Prevention/ | | | | |
| | UTI prevention | | | | |
| Lactobacilli: acidophilus, | Micro-biome | <6 billion | <6 billion | <6 billion | <6 billion |
| brevis, bulgaricus, | support | CFUs, 6 | CFUs, 6 | CFUs, 6 | CFUs, 6 |
| plantarum, rhamnosus, | | billion- 90 | billion- 90 | billion- 90 | billion- 90 |
| gasseri, casei, | | billion CFUs, | billion CFUs, | billion CFUs, | billion CFUs, |
| salivarius, paracasei, | | 90 billion + | 90 billion + | 90 billion + | 90 billion + |
| rueten, fermentum, | | CFUs. | CFUs. | CFUs. | CFUs. |
| breve, animalis, sreuteri, | | | | | |
| helvecticus, | | | | | |
| kefiranofaciens, | | | | | |
| kefigranum, lactis, | | | | | |
| cremoirs, themophilus, | | | | | |
| kefiri, parakefir, | | | | | |
| lactisbiovardiacetylactis, | | | | | |
| faceium, crispatus, | | | | | |
| iners, jensienii, | | | | | |
| sporagenus, delbrueckii; | | | | | |
| and Bifidobacteria, | | | | | |
| breve, bifidum, infantis, | | | | | |
| lactis, longum. | | | | | |
| Coconut oil, and or coco | Binder | 1-5 grams | 1-5 grams | 1-5 grams | 1-5 grams |
| butter, and or palm oil | | | | | |
| Urethral (bougies) | | Males: < or = | Males: < or = | Males: < or = | Males: < or = |
| (overall weight and | | 4 grams | 4 grams | 4 grams | 4 grams |
| dimensions) | | each, 100- | each, 100- | each, 100- | each, 100- |
| | | 150 mm long. | 150 mm long. | 150 mm long. | 150 mm long. |
| | | Females: < | Females: < | Females: < | Females: < |
| | | or = 2 grams | or = 2 grams | or = 2 grams | or = 2 grams |
| | | each and 60- | each and 60- | each and 60- | each and 60- |
| | | 75 mm in | 75 mm in | 75 mm in | 75 mm in |
| | | length | length | length | length |

### Aural and Nasal Formulation

Aural formulations may be used to decrease symptoms associated with mental disorders, including but not limited to intellectual disabilities, communication disorders, autism spectrum Disorder, Attention-deficit/Hyperactivity disorder, motor disorders and other neurodevelopmental disorders, schizophrenia spectrum and other psychotic disorders including PTSD (DSM-5, DSM-6).

TABLE 8
Formations 1-4 for aural/nasal use

| Ingredient | Function | Amount F1 | Amount F2 | Amount F3 | Amount F4 |
|---|---|---|---|---|---|
| CBGA/CBG, CBD | Anti- inflammatory, | <50 | 50-100 | 100-400 | 400+ |
| | analgesic, immune | | | | |
| | support | | | | |
| THCA/THC | anti- inflammatory, | Trace- | 50-100 | 100-400 | 400+ |
| | analgesic | <50 mg | | | |
| L- Acidophilus, L- | Microbiome support. | <6 billion | <6 billion | <6 billion | <6 billion |
| Plantarum, L- | | CFUs, 6 | CFUs, 6 | CFUs, 6 | CFUs, 6 |
| Rueten, S. | | billion- 90 | billion- 90 | billion- 90 | billion- 90 |
| Salvarius | | billion | billion CFUs, | billion | billion |
| | | CFUs, 90 | 90 billion + | CFUs, 90 | CFUs, 90 |
| | | billion + | CFUs. | billion + | billion + |
| | | CFUs. | | CFUs. | CFUs. |
| Psilocybin, | “bliss molecule” | Trace to | Trace to | Trace to | Trace to |
| MDMA | Antifungals/ | <5 mg | 45 mg | 45 mg | 45 mg |
| | antibacterials | | | | |
| Theobroma oil, | Binder | <1 to 5 | <1 to 5 | <1 to 5 | <1 to 5 |
| and or Coconut | | grams | grams | grams | grams |
| oil, and or | | | | | |
| cocobutter, and or | | | | | |
| palm oil | | | | | |
| Dimensions | | Nasal, < or = | Nasal, < or = | Nasal, < or = | Nasal, < or = |
| | | 1.2 grams, | 1.2 grams, | 1.2 grams, | 1.2 grams, |
| | | and <9 mm- | and <9 mm- | and <9 mm- | and <9 mm- |
| | | 10 mm long | 10 mm long | 10 mm long | 10 mm long |

### Prophetic Experiment

A vaginal suppository named Statera Stabilizer-V is compared for efficacy in the treatment of vaginal yeast infections to standards of care. A randomized study of a small cohort of women is conducted comparing Statera Stabilizer V to OTC Miconazole 3 and 7 day treatments. Patients will have benchmark testing and provide qualitative measurement of pain and other discomfort such as itching and swelling. Vaginal specimens will be obtained pre- and post-treatment and cell counts obtained.

We predict that Statera will outperform standards of care on resolution of candidiasis colonization and symptomatic decrease of pain and itching. Statera is expected to show complete resolution by day 7 vs. Miconazole alone (intra-vaginal and external cream). Moreover, the vaginal flora will be re-established more effectively and <5% will have recurrence within 6 months. Each ingredient in Statera serves regional purpose in re-establishing flora (probiotic delivery that does not need to go through the GI tract), and cannabinoid support for pain and inflammation. These in tandem, along with an anti-fungal, not only treat the vaginal yeast infection more effectively, but re-establishes healthy vaginal flora more effectively.

### Prophetic Experiment

We will repeat the above experiment with probiotics alone, cannabinoids alone and a combined formulation. We predict that the experiment will demonstrate that the combined effect of the two active ingredients and modality will be better than additive of each ingredient used alone, thus proving their synergistic effects.

The following cites are each incorporated by reference in their entireties for all purposes.

acslabcannabis.com/blog/education/a-guide-to-thc-delta-8-extraction-storage-price-consumption-and-more/

cancer.gov/publications/dictionaries/cancer-drug/def/delta-8-tetrahydrocannabinol?redirect=true

hempgazette.com/medical-cannabis/cannabinoids-list/

https://simplycbdandme.com/cannabis-terpenes-list-chart/

pubchem.ncbi.nlm.nih.gov/compound/delta8-THC

sciencedirect.com/topics/medicine-and-dentistry/dialister

who.int/medicines/access/controlled-substances/IsomersTHC.pdf

Amabebe E. & Anumba, D. O. C., The Vaginal Microenvironment: The Physiologic Role of Lactobacilli, Frontiers in Medicine 5:181 (2018), available online at frontiersin.org/article/10.3389/fmed.2018.00181

Atakan Z. Cannabis, a complex plant: different compounds and different effects on individuals. Ther Adv Psychopharmacol., 2(6):241-254 (2012), available online at ncbi.nlm.nih.gov/pmc/articles/PMC3736954/

Bedarf, J. R., et al. Functional implications of microbial and viral gut metagenome changes in early stage L-DOPA-naive Parkinson's disease patients. Genome Med 9, 39 (2017), available online at pubmed.ncbi.nlm.nih.gov/28449715/

Borgogna, J. L. C. & Yeoman, C. J., Chapter 3—The Application of Molecular Methods Towards an Understanding of the Role of the Vaginal Microbiome in Health and Disease, Methods in Microbiology 44: 37-91 (2017), available online at sciencedirect.com/science/article/pii/S058095171730003X

Brenneisen R. Chemistry and Analysis of Phytocannabinoids and Other Cannabis Constituents. In: ElSohly M. A. (eds) Marijuana and the Cannabinoids. Forensic Science And Medicine (Humana Press 2007). Available online at nature.com/articles/srep24380.

Dobritsa A. P., et al., Reclassification of Eubacterium combesii and discrepancies in the nomenclature of botulinum neurotoxin-producing clostridia: Challenging Opinion 69. Request for an Opinion. Int J Syst Evol Microbiol. 2018 September; 68(9):3068-3075 (2018), available online at doi: 10.1099/ijsem.0.002942.

Goya, M. E., et al., Probiotic Bacillus subtilis Protects against α-Synuclein Aggregation in C. elegans. Cell Rep. 14; 30(2):367-380.e7 (2020), available online at pubmed.ncbi.nlm.nih.gov/31940482/

Petrov, V. A., et al. Analysis of Gut Microbiota in Patients with Parkinson's Disease. Bull Exp Biol Med 162, 734-737 (2017), available online at doi.org/10.1007/s10517-017-3700-7

Melis M, Pistis M. Endocannabinoid signaling in midbrain dopamine neurons: more than physiology?. Curr Neuropharmacol., 5(4):268-277 (2007), available online at ncbi.nlm.nih.gov/pmc/articles/PMC2644494/

Sakakibara R., et al., Lewy body constipation, J Anus Rectum Colon 3(1):10-17 (2019), available online at ncbi.nlm.nih.gov/pmc/articles/PMC6752132/

Shen, J., Song, N., Williams, C. et al. Effects of low dose estrogen therapy on the vaginal microbiomes of women with atrophic vaginitis. Sci Rep 6, 24380 (2016), available online at nature.com/articles/srep24380.

Smith R. J. & Bryant R G. Metal substitutions incarbonic anhydrase: a halide ion probe study. Biochem Biophys Res Commun. 1975 Oct. 27; 66(4):1281-6 (1975), available online at pubmed.ncbi.nlm.nih.gov/3/

Stefanis, L., α-Synuclein in Parkinson's disease, Cold Spring Barb Perspect Med. 2(2):a009399 (2012), available online at ncbi.nlm.nih.gov/pmc/articles/PMC3281589/

Valles-Colomer, M., et al. The neuroactive potential of the human gut microbiota in quality of life and depression. Nat Microbiol 4, 623-632 (2019), available online at nature.com/articles/s41564-018-0337-x

Yeager, A., Specific Gut Microbes Linked with Depression: Study, The Scientist (2019), available online at http://www.the-scientist.com/news-opinion/specific-gut-microbes-linked-with-depression-study-65427#:˜:text=Two%20types%20of%20bacteria.act%20on%20the%20nervous%20system

US20110086113 Cannabinoids in combination with non-cannabinoid chemotherapeutic agents

US20120237489 Suppository for rectal, vaginal or urethral administration containing a probiotic, an antibiotic and an unsaturated non-esterified fatty acid

US20160136127 Use of tetrahydrocannabinol and/or cannabidiol for increasing radiosensitvity in the treatment of a brain tumour

US20170173091 Probiotic delivery systems

U.S. Pat. No. 5,176,911 Pharmaceutical compositions comprising selected Lactobacillus strains

U.S. Pat. No. 6,180,100 Lactobacillus compositions and methods for treating urinary tract infections

U.S. Pat. No. 6,277,370 Pharmaceutical compositions containing Lactobacilli for treatment of vaginal infections and related method

U.S. Pat. No. 6,410,588 Use of cannabinoids as anti-inflammatory agents

U.S. Pat. No. 8,846,029 Treatment of IBD and IBS using both probiotic bacteria and fermented cereal as treatment effectors

U.S. Pat. No. 9,050,358 Compositions and methods for probiotic recolonization therapies

U.S. Pat. No. 9,421,187 Phytocannabinoids for use in the treatment of intestinal inflammatory diseases

WO2009043836 A composition containing non-psychotropic cannabinoids for the treatment of inflammatory diseases

1. A suppository formulation for the local treatment of infection or inflammation or both, said formulation comprising:
a) a base that is a solid at room temperature and that liquifies or dissolves at about 37° C. in a human body;
b) one or more probiotics dispersed in said base;
c) one or more cannabinoids dispersed in said base; and
d) said formulation being in the format of a solid suppository.

2. The formulation of claim 1, said cannabinoid being at 50-500 mg per dose.

3. The formulation of claim 1, said probiotic being at 1 billion to 100 billion CFUs per dose.

4. The formulation of claim 1, said cannabinoid being at CBD and THC at about a 1 to 1 ratio.

5. The formulation of claim 1, said base comprising a hard fat being selected from coco butter, coconut oil, or palm oil.

6. The formulation of claim 1 for use in the vagina, said probiotic selected from the group consisting of Lactobacillus, Bifidobacteria, and Lactococcus and said cannabinoid selected from the group consisting of CBGA, CBG, CBD, THCA, THC, THCVA, and THCV.

7. The formulation of claim 1 for use in the vagina, said probiotic selected from the group consisting of Lactobacilli: acidophilus, brevis, bulgaricus, plantarum, rhamnosus, gasseri, casei, salivarius, paracasei, fermentum, salivarius, reuteri, helveticus, lactis, cremoris, kefiri, parakefir, faecium, crispatus, delbrueckii, and Lactococcus lactus, and said cannabinoid selected from the group consisting of CBGA, CBG, CBD, THCA, THC, THCVA and THCV.

8. The formulation of claim 1 for use in the rectum, said probiotic selected from the group consisting of Lactobacillus, Lactococcus, and Bifidobacteria, and said cannabinoid selected from the group consisting of CBGA, CBG, CBD, TCHA, THC, THCVA, and THCV.

9. The formulation of claim 1 for use in the rectum, said probiotic selected from the group consisting of Lactobacilli: acidophilus, brevis, bulgaricus, plantarum, rhamnosus, gasseri, casei, salivarius, paracasei, rueten, fermentum, breve, animalis, sreuteri, helvecticus, kefiranofaciens, kefigranum, lactis, cremoirs, themophilus, kefiri, parakefir, lactisbiovardiacetylactis, faceium, crispatus, iners, jensienii, sporagenus, and delbrueckii and Bifidobacteria, breve, bifidum, infantis, lactis, and longum, and said cannabinoid selected from the group consisting of: CBGA, CBG, THCA, THC, CBDA, CBD, CBCA, CBC, CBVA, THCV, CBDVA, CBDV, CBN, CBNA, and CBNA.

10. The formulation of claim 1, said formulation being i) CBGA, CBG, THCA, THC, CBDA-CBD, ii) Lactobacilli: acidophilus, plantarum, casei, breve, and brevis, iii) Bifidobacteria: breve, bifidum, and lactis, and iv) boric acid.

11. The formulation of claim 6, said formulation being i) Lactobacilli: acidophilus, brevis, bulgaricus, plantarum, rhamnosus, gasseri, casei, salivarius, paracasei, fermentum, salivarius, reuteri, helveticus, lactis, cremoris, kefiri, parakefir, faecium, crispatus, and delbrueckii, ii) Lactococcus lactus, and iii) CBGA, CBG, CBD, THCA, THC, THCVA and THCV.

12. The formulation of claim 8, said formation being i) Lactobacilli: acidophilus, brevis, bulgaricus, plantarum, rhamnosus, gasseri, casei, salivarius, paracasei, rueten, fermentum, breve, animalis, sreuteri, helvecticus, kefiranofaciens, kefigranum, lactis, cremoirs, themophilus, kefiri, parakefir, lactisbiovardiacetylactis, faceium, crispatus, iners, jensienii, sporagenus, and delbrueckii: ii) Bifidobacteria, breve, bifidum, infantis, lactis, and longum, and iii) said cannabinoid selected from the group consisting of: CBGA, CBG, THCA, THC, CBDA, CBD, CBCA, CBC, CBVA, THCV, CBDVA, CBDV, CBCV, CBN, and CBNA.

13. A method of treating a vaginal infection or inflammation, said comprising administering a suppository having the suppository formulation of claim 1 to a vagina having an infection or inflammation at least daily for at least three days.

14. A method of treating a vaginal infection or inflammation, said comprising administering a suppository having the suppository formulation of claim 6 to a vagina having an infection or inflammation at least daily for at least three days.

15. A method of treating a gastrointestinal disease, said comprising administering a suppository having the suppository formulation of claim 1 to a rectum having a gastrointestinal disease at least daily for at least three days.

16. A method of treating a gastrointestinal disease, said comprising administering a suppository having the suppository formulation of claim 8 to a rectum having a gastrointestinal disease at least daily for at least three days.

17. A method of treating an aural or nasal condition said comprising administering a suppository having the suppository formulation of claim 1 to an ear or nose at least daily for at least three days.

18. A method of treating a urogenital infection or inflammation, said comprising administering a suppository having the suppository formulation of claim 1 to a urethra having an infection or inflammation at least daily for at least three days.

19. The formulation of claim 1, said cannabinoid being selected from the group consisting of Cannabichromene (CBC), Cannabichromenic acid (CBCA), Cannabichromevarin (CBCV), Cannabichromevarinic acid (CBCVA), Cannabidihexol (CBDH), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CRNA), Cannabiorcool (CBN-C1), Cannabivarin (CBV), Cannabichromevarin (CBCV), 10-Ethoxy-9-hydroxy-delta-6a-tetrahydrocannabinol, 8,9-Dihydroxy-delta-6a-tetrahydrocannabinol, Cannabitriol (CBT), Cannabitriolvarin (CBTV), Delta-8-tetrahydrocannabinol (A8-THC), Delta-8-tetrahydrocannabinolic acid (A8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC), and 3,4,5,6-Tetrahydro-7-hydroxy-alpha-alpha-2-trimethyl-9-n-propyl-2,6-methano-2H-1-benzoxocin-5-methanol, (OH-iso-HHCV).

20. The formulation of claim 1, wherein said cannabinoid is dispersed in a first portion of said base and said probiotic is dispersed in a second portion of said base.
